(19) United States
(12) Patent Application Publication
(10) Pub. No.: US 2005/0255097 A1
(43) Pub. Date: Nov. 17, 2005

(54) Modified venom and venom components as anti-retroviral agents

(76) Inventors: Paul Reid, (US); Laurence Raymond, (US)

Correspondence Address: ROBERT J. VAN DER WALL, 1200 Brickell Avenue, Suite 1620, Miami, FL, 33131, US

(21) Appl. No.: 10/764,356

(22) Filed: Jan. 26, 2004

(51) Int. Cl. A61K 38/48; A61K 38/16

(52) U.S. Cl. 424/94.64; 514/2

(57) ABSTRACT

The present invention relates to a class of proteins, a process of production thereof, and a method for treatment of neurological and viral diseases in humans and animals. More specifically it applies to the treatment of heretofore intractable diseases such as retro-viral infections including human immunodeficiency virus (HIV), simian immunodeficiency virus (SIV), feline immunodeficiency virus (FIV), bovine immunodeficiency virus (BIV) and equine acquired immunodeficiency virus (EAIV). The

### BACKGROUND OF THE INVENTION

1. Field of the Invention

The present invention relates to a class of proteins, a process of production thereof, and a method for treatment of neurological and viral diseases and especially to the treatment of heretofore intractable diseases such as retro-viral infections, including specifically HIV infections.

2. Description of Prior Art

Sanders, et al. had commenced investigating the application of modified venoms to the treatment of ALS in 1953 having employed poliomyelitis infection in monkeys as a model. Others antiviral studies had reported inhibition of pseudorabies (a herpesvirus) and Semliki Forest virus (alpha-virus). See Sanders' U.S. Pat. Nos. 3,888,977, 4,126,676, and 4,162,303. Sanders justified the pursuit of this line of research through reference to the studies of Lamb and Hunter (1904) though it is believed that the original idea was postulated by Haast. See Haast U.S. Pat. Nos. 4,341,762 and 4,741,902. See also MacDonald, et al., U.S. Pat. No. 5,723,477. The studies of Lamb and Hunter (Lancet 1:20, 1904) showed by histopathologic experiments with primates killed by neurotoxic Indian cobra venom that essentially all of the motor nerve cells in the central nervous system were involved by this venom. A basis of Sanders' invention was the discovery that such neurotropic snake venom, in an essentially non-toxic state, also could reach that same broad spectrum of motor nerve cells and block or interfere with invading pathogenic bacteria, viruses or proteins with potentially deleterious functions. Thus, the snake venom used in producing the composition was a neurotoxic venom, i.e., causing death through neuromuscular blockade. As the dosages of venom required to block the nerve cell receptors would have been far more than sufficient to quickly kill the patient, it was imperative that the venom was detoxified. The detoxified but undenatured venom was referred to as being neurotropic. The venom was preferably detoxified in the mildest and most gentle manner. While various detoxification procedures were known then to the art, such as treatment with formaldehyde, fluorescein dyes, ultraviolet light, ozone, heat, it was preferred that gentle oxygenation at relatively low temperatures be practiced, although the particular detoxification procedure was not defined as critical. Sanders employed a modified Boquet detoxification procedure using hydrogen peroxide, outlined below. The acceptability of any particular detoxification procedure was tested by the classical Semliki Forest virus test, as taught by Sanders, U.S. Pat. No. 4,162,303.

U.S. Pat. No. 3,888,977, issued on Jun. 10, 1975 to Murray J. Sanders (the entire disclosure of which is incorporated herein by reference and relied upon for details of disclosure) teaches that animals, including humans, may be treated for progressive degenerative neurological diseases, such as amyotropic lateral sclerosis, by administration of a modified snake venom neurotoxin derived from the venom of either the Bungarus genus (including the Crotalus genus) or from a combination of the Bungarus genus and the Naja genus, i.e., in either case the therapeutic composition must contain at least in part modified neurotoxin derived from the Bungarus genus. Thus, it is taught that while the Bungarus venom can be effectively used alone, the Naja venom must be used in combination with the Bungarus venom. Unfortunately, however, Bungarus venom is not as readily available as Naja venom; the supply thereof is more uncertain; and it is far more expensive than the Naja venom. Sanders U.S. Pat. No. 4,126,676 (1978) provided a method of treatment of animals suffering from progressive degenerative neurological diseases wherein the therapeutic modified neurotoxin was derived from the Naja genus alone. Miller, et al. (1977) reported that the modified venoms antiviral activity against Semliki Forest virus was associated with several chromatographic fractions comprising the neurotoxic components. The most abundant component with antiviral activity was shown to be alpha-cobratoxin. Yourist, et al. (1983) reported that modified alpha-cobratoxin could inhibit the activity of herpesvirus. It seemed therefore, that these modified venoms and constituents had significant inhibitory activity against unrelated viruses. This non-specific activity has prompted the examination of these modified venom products against a number of viral types.

Other references of interest include four patents, Haast, U.S. Pat. Nos. 4,741,902 and 5,723,477, Hoxie, U.S. Pat. No. 5,994,515 and Au-Yuong, et al., U.S. Pat. No. 5,955,303. Literature references of interest are: Battaglioli E., Gotti C., Terzano S., Flora A., Clementi F. and Formasari D.; J. Neurochem. 71: 1261-1270 (1998), Benhammou K., Lee M., Strook M., Sullivan B., Logel J., Raschen K., Gotti C. and Leonard S.; Neuro-pharmacology 39: 2818-2829 (2000), Bewley C. A. and Otero-Quintero S.; J. Am. Chem. Soc. 123: 3892-3902 (2001), Boquet P.; Ann. Inst. Pasteur 66: 379-396 (1941), Boyd M. R., Gustafson K. R., McMahon J. B., Shoemaker R. H., O'Keefe B. R., Mori T., Gulakowski R. J., Brockes J. P. and Hall Z. W.; Biochemistry 14: 2092-2099 (1975), Boyle M. J., Conners M., Flanigan M. E., Geiger S. P., Ford H. Jr., Baseler M., Adelsberger J., Davey R. T. Jr., Lane H. C.; J. Immunol. 154: 6612-6623 (1995), Bhattacharya T.; Science 288: 1789-1796 (2000), Bonyhadi M. L., Su L., Auten J., McCune, J. M., Kaneshima, H.; AIDS Res. & Hum. Retroviruses 11: 1073-1080 (1995), Bracci L., Lozzi L., Rustici M. and Neri P.; FEBS 311: 115-118 (1992), Bracci L., Ballas S. K., Spreafico A. and Neri P.; Blood 90: 3623-3628 (1997), Cammack N.; Curr. Opin. Infect. Dis. 14: 13-16 (2001)], Chang L. C. and Bewley CA.; J. Mol. Biol.: 318: 1-8 (2002), Chang C. C., Kawata Y., Sakiyama F. and Hayashi K.; Eur. J. Biochem. 193: L567-572 (1990), Choe H., Farzan M., Sun Y., Sullivan N., Rollins B., Ponath P. D., Wu L., Mackay C. R., LaRosa G., Newman W., Gerard N., Gerard C. and Sodroski J.; Cell 85: 1135-1148 (1996), Collins K. B., Patterson B. K., Naus G. J., Landers D. V., Gupta P.; Nature Med. 6: 475-479 (2000), Courgnaud V., Pourrut X., Bibollet-Ruche F., Mpoudi-Ngole E., Bourgeois A., Delaporte E. and Peeters M.; J. Virol. 75: 857-866 (2001), Davies B. D., Hoss W., Lin J. P. and Lionetti F.; Mol. Cell Biochem. 44: 23-31 (1982), Derdeyn C. A., Decker J. M., Sfakianos J. N., Wu X., O'Brien W. A., Ratner L, Kappes J. C., Shaw G. M., and Hunter E; J. Virol. 74: 8358-8367 (2000), De Clerque E.; Mini. Rev. Med. Chem. 2: 163-175 (2002), Deng H., Liu R., Ellmeier W., Choe S., Unutmaz D., Burkhart M., diMarzio P., Marmaon S., Sutton R. E., Hill C. M., Davis C. B., Peiper S. C., Schall T. J., Littman D. R. and Landau N. R.; Nature 381: 661-666 (1996), Dowding A. J. and Hall Z. W.; Biochemistry 26: 6372-6381 (1987), D'Souza M. P., Cairns J. S. and Plaeger S. F.; J.A.M.A. 284: 215-222 (2000), Esser M. T., Mori T., Mondor I, Sattentau Q. J., Dey B, Berger E. A., Boyd M. R. and Lifson J. D.; J. Virol. 73: 4360-4371(1999), Feng Y., Broder C. C., Kennedy P. E. and Berger E. A.; Science 272: 872-877 (1996), Franti M., O'Neill, Maddon P., Burton D. R., Poignard P. and Olson W.; 9^{th} Conference on retroviruses and opportunistic infections; February 24-28, Washington State Convention Trade Center, Seattle Wash.; 2002., Froehner S. C. and Rafto S.; Biochemistry 18: 301-307 (1979), Fujii T., Tsuchiya T., Yamada S., Fujimoto K., Suzuki T., Kasahara T. and Kawashima K.; J. Neurosci. Res. 44: 66-72 (1996), Fujii T. and Kawashima K.; Jpn. J. Pharmacol. 85: 11-15 (2001), Greenhead P., Hayes P., Watts P. S., Laing K. G., Griffin G. E. and Shattock R. J.; J. Virol. 74: 5577-5586 (2000), Gustafson K. R., Sowder R. C., Henderson L. F., Cardellina J. H., McMahon J. B., Rajamani U., Pannell L. K. and Boyd M. R.; Biochem. Biophys. Res. Commun. 238: 223-228 (1997), Grabczewska E., Laskowska-Bozek H., Maslinski W. and Ryzewski J.; Int. J. Tissue React. 12: 281-289 (1990), Hallquist N., Hakki A., Wecker L., Friedman H. and Pross S.; Proc. Soc. Exp. Biol. Med. 224: 141-146 (2000), Hanna S. L., Yang C., Owen S. M. and Lal. R. B.; AIDS 16: 1603-1608 (2002), Hiemke C., Stolp M., Reuss S., Wevers A., Reinhardt S., Maelicke A., Schlegel S. and Schroder H.; Neurosci. Lett. 214:171-174 (1996), Horn T. and Braun J. F.; P.R.N. Notebook: http://www.prn.org_nb_cntnt/cap08-15-02.06.htm, Jiang S., Zhao Q. and Debnath A. K.; Curr. Pharm. Des. 8: 563-580 (2002), Kawashima K. and Fujii T.; Pharmacol. Ther. 86: 29-48 (2000), Kawashima K., Fujii T., Watanabe Y. and Misawa H.; Life Sci. 62: 1701-1705 (1998), Kilby J. M., Hopkins S., Venetta T. M., DiMassimo B., Cloud G. A., Lee J. Y., Alldredge L., Hunter E., Lambert D., Bolognesi D., Matthews T., Johnson M. R., Nowak M. A., Shaw G. M. and Saag M. S.; Nat. Med. 4: 1302-1307 (1998), Kolchinsky P., Kiprilov E., Bartley P., Rubinstein R. and Sodroski J.; J. Virol. 75: 3435-3443 (2001), Korber B., Muldoon M., Theiler J., Gao F., Gupta R., Lapedes A., Hahn B. H., Wolinsky S., Bhattacharya T.; Science 288: 1787-1796 (2000), Koyanagi Y., Tanaka Y., Kira J., Ito M., Hioki K., et al.; J. Virology 71: 2417-2424 (1997), Lamb G. and Hunter, W. K., Lancet, 1: 20-22 (1904), Lentz T. L., Burrage T. G., Smith A. L., Crick J. and Tigor G. H.; Science 215: 182-184 (1982), Lenz T. L., Hawrot E. and Wilson P. T.; Proteins:Structure, Function and Genetics 2: 298-307 (1987), Levin J.; Report; 42^{nd} ICAAC Meeting, San Diego, Sept. 27-31 (2002); Mariner J. M., McMahon J. B., O'Keefe B. R., Nagashima K. and Boyd M. R.; Biochem. Biophys. Res. Commun. 30: 841-845 (1998), Markham R. B., Schwartz D. H., Templeton A., Margolick J. B., Farzadegan H., et al.; J. Virology 70:6947-6954 (1996), McLane K. E., Fritzen M., Wu X., Diethelm B., Maelicke A. and Conti-Tronconi B. M; J. Recept. Res. 12: 299-321 (1992), McLeod G. X., McGrath J. M., Ladd E. A., Hammer, S. M.; Antimicrob. Agents Chemother. 36: 920-925 (1992), Miller K. D., Miller G. G. and Sanders M., Fellows O. N.; Biochem. Biophys. Acta 496: 192-196 (1977), Mizuno Y., Dosch H. M. and Gelgand E. W.; J. Clin. Immunol. 2: 303-308 (1982), Moore J. P., Sattentau Q. J., Wyatt R. and Sodroski J.; J. Virol. 68: 469-484 (1994), Mori T. and Boyd M. R; Antimicro. Agents Chemother. 45: 664-672 (2001), Myers G. and Lu H.; http://hiv-web.lanl.gov/content/hiv-db/REVIEWS/articles Nagashima K. A., Thompson D. A., Rosenfield S. I., Maddon P. J., Dragic T. and Olson W. C.; J. Infect. Dis. 183: 1121-1125 (2001), Neri P., Bracci L., Rustici M. and Santucci A.; Arch. Virol. 114: 265-269 (1990), Patterson, B., Flener, Z., Yogev, R. and Kabat, W. “Inhibition of HIV-1 replication in mononuclear cells and thymus explant cultures by a purified, detoxified cobra venom protein” (2000) Abstract, “Novel biological fusion inhibitors of HIV”, Apr. 7, 2000, Keystone Conference, Colorado., Peters B. S.; Antivir. Chem. Chemother. 11: 311-320 (2000), Piot P.; Science 280: 1844-1845 (1998) Reeves J., Puffer B., Ahmad N., Derdeyn C., Sharron M., Edwards T., Carlin D., Harvey P., Pierson T., Hunter E; and Doms R. W.; 9^{th} Conference on retroviruses and opportunistic infections; Feb. 24-28, Washington State Convention Trade Center, Seattle Wash.; 2002., Rusconi S., Moonis M., Merrill D., Pallai P. V., Neidhardt E. A., Singh S. K., Willis K. J., Osburne M. S., Profy A. T., Jenson J. C. and Hirsch M. S.; Antimicrobial Agents and Chemotherapy 40: 234-236 (1996), Schearer W. T., Israel R. J., Starr S., Fletcher C. V., Wara D., Rathore M., Church J., DeVille J., Fenton T., Graham B., Samson P., Staprans S., McNamara J., Moye J., Maddon P. J. and Olson W. C.; J. Infect. Dis. 182: 1774-1779 (2000), Sato K. Z., Fujii T., Watanabe Y., Yamada S., Ando T., Kazuko F. and Kawashima K.; Neurosci. Lett. 26617-20 (1999), Sanders M. and Fellows O.; (1974) In Excerpta Medica; International Congress Series No. 334 containing abstracts of papers presented at the III International Congress of Muscle Diseases, Newcastle on Tyne, September, Sanders, M., Soret, M. G. and Akin, B. A.; Ann. N.Y. Acad. Sci. 53: 1-12 (1953), Sanders, M., Soret, M. G., and Akin, B. A.; J. Path. Bacteriol. 68: 267-271 (1954), Sanders, M., Soret M. G. and Akin B. A.; J. Path. Bact. 68: 267-271 (1954a), Sanders, M., Soret M. G. and Akin B. A.; Acta Neurovegetat 8: 326-327 (1954b) Sanders, M., Soret M. G. and Akin B. A., Roizin L.; Science 127: 594-596 (1958a), Sanders, M., Soret M. G. and Akin B. A.; Proc 7^{th} Inter. Cong. Microbiol.; p. 293 (1958b), Sanders, M. and Fellows O.; Cancer Cytology 15: 34-40(1975) Schols D., Este J. A., Henson G. and Declerq E.; Antiviral Res. 35: 147-156 (1997), Schols D., Claes S., De Clercq E., Hendrix C., Bridger G., Calandra G., Henson G., Fransen S., Huang W., Whitcomb J. M. and Petropoulos J; 9^{th} Conference of retroviruses and opportunistic infections; (2002), Singh S. P., Karla R., Puttfarcken P., Kozak A., Tesfaigzi J. and Sopori; Toxicol. Appl. Pharmacol. 164: 65-72 (2000), Starcich B. R., Hahn B. H., Shaw G. M., McNeely P. D., Modrow S, Wolf H., Parks E. S., Parks W. P., Josephs S. F. and Gallo R. C.,; Cell 45: 637-648 (1986), Sullivan N., Sun Y., Sattentau Q., Thali M., Wu D., Denisova G., Gershoni J., Robinson J., Moore J., and Sodroski J.; J. Virol. 72: 4694-4703 (1998), Thali M., Moore J. P., Furman C., Charles M., Ho C. C., Robinson J. and Sodroski J.; J. Virol. 67: 3978-3988 (1993), Toyabe S., Iiai T., Fukuda M., Kawamura T., Suzuki S., Uchiyama M. and Ado T.; Immunology 92: 201-205 (1997), Tremblay C. L., Kollmann C., Giguel F., Chou T. C. and Hirsch M. S.; J. Acquir. Immune Defic. Syndr. 25: 99-102 (2000), Tu A. T.; Ann. Rev. Biochem. 42: 235-258(1973) VanDamme L., Wright A., Depraetere K., Rosenstein I., Vandermissen V., Poulter L., McKinlay M., Van Dyck E., Weber J., Profy A., Laga M. and Kitchen V.; Sex. Transm. Infect. 76: 126-130 (2000), WeberJ., NunnA., O'Conner T., Jeffries D., Kitchen V., McCormack S., Stott J., Almond N., Stone A. and Darbyshire J.; AIDS 15: 1563-1568 (2001), Wei X., Decker J. M., Liu H., Zhang Z., Arani R. B., Kilby J. M., Saag M. S., Wu X., Shaw G. M. and Kappes J. C.; Antimicrob. Agents Chemother. 46: 1896-1905 (2002), Wild C. T., Shugars D. C., Greenwell T. K., McDanal C. B. and Matthews T. J.; Proc. Natl. Acad. Sci. 91: 9770-9774 (1994), Wu L., Gerard N. P., Wyatt R., Choe H., Parolin C., Ruffing N., Borsetti A., Cardoso A. A., Desardin E., Newman W. and Sodroski J.; Nature 384: 179-183(1996), Wu L., Rivera M. I., Laurencot C. M., Currens M. J., Cardellina J. H., Buckheit R. W., Nara P. L., Pannell L. K., Sowder R. C. and Henderson L. E.; Antimicro. Agents and Chemotherapy 41: 1521-1530 (1997), Wyatt R., Moore J., Accola M., Desjardin E., Robinson J. and Sodroski J.; J. Virol. 69: 5723-5733 (1995), Zhu C. B., Zhu L., Holz-Smith S., Matthews T. J. and Chen C. H.; PNAS 98: 15227-15232 (2001).

### SUMMARY OF THE INVENTION

The present invention provides a composition and method for treating and preventing retroviral infections of mammalian cells. One aspect of the invention relates to the identification of modified neurotoxins capable of preventing HIV infection and replication in that cell. In another aspect the invention relates to an retroviral composition derived from modified venom which can be administered in-vivo for the treatment of HIV infection. In another aspect, the invention relates to the synergistic effects of modified venom constituents in preventing HIV infection and replication. In another aspect, the retrovirus is selected from the group consisting of Lentiviruses (HIV-1, HIV-2, SIV, EIAV, BIV, and FIV).

Proteins such as those from venoms, as described herein, have long been recognized for their ability to bind to specific receptors on the surface of mammalian cells. These neurospecific proteins bind to such common receptors as the acetylcholine receptor for example. However, the protein motif employed by these neurotoxins to affect binding appears to be a common motif employed by other, apparently unrelated, proteins including those present in viral coat proteins. Such viral proteins include rabies virus coat protein and gp120 from HIV. Prior studies had indicated that proteins with these motifs could interfere with the activity of the other. Sanders provided a method which permits the safe administration of venom proteins allowing the application of these laboratory observations to practical use. Therefore included in the invention is a method of treating lentivirus infection in mammals and humans comprising administering to the host of either the modified venom or the modified neurotoxin.

In yet another aspect of the invention is the indication that modified neurotoxins can bind to a HIV receptor protein and/or a cellular cofactor unrelated to their original target receptors. The specific entity to which MCTX binds is presently unknown, though it appears to have an impact upon viral infection late in infection, possibly during maturation of infectious particles.

In another aspect of the invention, the modified venom's higher antiviral activity suggests the existence of synergism between venom components due to the presence of other neurotoxic components in addition of alpha neurotoxin known as cobratoxin. Therefore, as a group consisting of modified alpha-neurotoxins with homologous domains and acetylcholine receptor binding activity can inhibit lentivirus infection and could be selected from but not limited to alpha-cobratoxin, alpha-bungarotoxin, alpha-cobrotoxin and alpha-conotoxin.

### DETAILED DESCRIPTION OF THE INVENTION

Although the survival of individuals currently infected by the HIV virus is dramatically longer than it was 20 years ago, such survival is at the cost of a drug regime which is highly expensive, complicated, relegated to a fixed time and sequence schedule, has adverse physiological side effects and is, ultimately, too little too late. While the logical method to halt the spread of the disease is sexual abstinence, such method embodies so many facets of world society, that, realistically, the disease will remain uncontrollable until such a time as it can be controlled by methods which are inexpensive, have few side effects, and can be administered easily.

Prophylaxis, utilized before or after potential exposure, fulfills these requirements. Potential prevention/treatment could take many forms; three are: 1. The development of a vaccine that prevents infection; 2. Prevention of an initial infection or control of the spread of an initial infection that has not progressed to AIDS by a means other than a vaccine, or, 3. A resolution of the syndrome known as AIDS by the use of anti-retroviral agents. While vaccine production is ultimately the most efficacious of the three methods, due to the mutational idiosyncrasies of the virus, such development is not a likely or a probable immediate occurrence. Vaccine development attempts to date have failed to translate into man from animal test-models (Peters; 2000).

Medical research resources are currently being applied to the management, rather than the cure of a HIV infection. While the use of anti-retrovirals agents have improved the quality and length of life, they have disadvantages which include toxicity, development of drug resistance, persistence of latently infected cells resulting in viral rebound after prolonged treatment and, finally, high expense. The prevention and/or control of an infection prior to loss of immune capabilities associated with progression to AIDS is currently the most expedient and cost effective method. Currently, there are several approved drugs types that apply themselves to the control of an ongoing HIV infection. These drug types are, nucleoside reverse transcriptase inhibitors, non-nucleoside reverse transcriptase inhibitors and protease inhibitors. These are currently encompassed by highly aggressive anti-retroviral therapy (HAART). All these drug types are susceptible to loss of effectiveness due to genetic mutation of the HIV-1. Thus, the blockade of HIV infection or the control of the spread of HIV infection through the use of fusion or entry inhibitors appears to be the most logical method barring the availability of a vaccine. Such blocking substance, or substances, could be applied topically, as a cream or douche, and provide protection during coitus. The use of this mode of prevention has been suggested by others (Turpin, 2002) and is being implemented (Van Damme, et al., 2000). The utilization of a binding/entry inhibitor as a prophylactic that would block infection and maintain a period of protection in the genital tract could provide an effective measure which would reduce HIV-1 transmission (D'Sousa, et al., 2000). Topical administration would not be amenable to prevention of disease by blood transfer by more direct routes (such as needles). However, as an injectable, or by buccal administration, it could be applicable parenterally in the treatment of an HIV infection during early stages of exposure, or later, by providing control of HIV dissemination within the host.

Alternatively, drug activities which alter the virus and reduce its infectivity or alter its functional form upon release would supply a mechanism for infection control at the “other side” of the infection sequence.

HIV-1 is a lentivirus (lenti=slow {Latin}) of the family Retroviridae. The virus is enveloped, 80-130 nm in diameter and has an icosahedral capsid. As with other lentiviruses, HIV can infect terminally differentiated, non-dividing cells such as macrophages resident in tissue or brain (microglia) as well as cells of the T cell lineage, specifically CD4+ cells, known as T helper (TH) cells. Lentiviruses have, through mutation, the capability to infect immune cells (macrophages; TH-cells), the ability to avoid immune system eradication and, thus, tend to persist for the life of their host. The typical HIV infection progresses through three stages: initial, or acute, associated with high levels of viral replication and dissemination, a latent stage attributed to partial immune system control, which is followed by the third stage which encompasses the return of high levels of viral replication and progression to clinical disease states due to decreased immunocompetence, termed acquired immunodeficiency syndrome (AIDS). HIV is suggested to be derived from the simian immunodeficiency virus (SIV) (Courgnaud, et al., 2001) and first entered the human population between 1915 and 1941 (Korber, et al., 2000). Two HIVs are associated with human AIDS: HIV-1 and HIV-2. HIV-1 is distributed worldwide and is responsible for the current AIDS pandemic while HIV-2 is currently restricted to West Africa. Both are spread by the same routes, though HIV-2 may be less pathogenic.

Treatment of HIV infection currently encompasses two basic modalities: drug action at host intracellular targets (post entry) and drug interaction at viral extracellular targets (pre-entry). The latter are termed as binding/entry inhibitors. Extracellular targets are those associated with viral attachment, fusion and entry into the host cell. Intracellular targets are those associated with viral nucleic acid synthesis and processing and are termed as anti-retroviral drugs. There are currently 16 licensed antiretroviral drugs employed to combat HIV-1 infection (D'Souza, et al. 2000, aidsmeds.com, 2002a). Currently, there is a drug, T-20 (Trimeris), which is licensed as a binding/entry inhibitor. Within the context of this proposal, extracellular targets are of immediate importance, consequently, discussions of viral inhibition post-cell entry will be omitted.

Infection by HIV occurs following the introduction of the virus to the blood of the potential host. Virus-host cell interaction is mediated through the viral envelope glycoproteins gp120 and gp41 (gp160), which are assembled as trimers on the surface of the viral envelope, and their interactions with host cell surface receptors CD4, and CXCR4 or CCR5. U.S. Pat. No. 5,994,515 (Hoxie) describes the manner in which the human immunodeficiency viruses HIV-1 and HIV-2 and the closely related simian immunodeficiency viruses (SIV), all use the CD4 molecule as a receptor during infection though viruses like HIV and FIV can infect CD4 negative cells. The latter two host cell surface receptors are chemokine receptors and act as co-receptors along with CD4. Chemokines are a large family of low molecular weight, inducible, secreted, proinflammatory cytokines which are produced by various cell types. See, for instance, Au-Yuong, et al., U.S. Pat. No. 5,955,303. Chemokines have been divided into several subfamilies on the basis of the positions of their conserved cysteines. The CC family includes monocyte chemoattractant protein-1 (MCP-1), RANTES (regulated on activation, normal T cell-expressed and secreted), macrophage inflammatory proteins (MIP-1.alpha., MIP-1.beta.), and eotaxin. (Proost, P. (1996) Int. J. Clin. Lab. Res. 26: 211-223; Raport, C. J. (1996) J. Biol. Chem. 271: 17161-17166). The CXC family includes interleukin-8 (IL-8), growth regulatory gene, neutrophil-activating peptide-2, and platelet factor 4 (PF-4). Although IL-8 and PF-4 are both polymorphonuclear chemo-attractants, angiogenesis is stimulated by IL-8 and inhibited by PF-4. However, the macrophage tropic (CCR5) strain BaL, is not capable of infecting cells which co-express both CXCR4 and CD4. These results suggest that CXCR4 can serve as a co-factor for T-tropic, but not M-tropic, HIV-1 strains (Feng, et al., 1996, supra). Moreover, the finding that there is a change from M to T-tropic viruses over time in infected individuals correlates with disease progression suggests that the ability of the viral envelope to interact with CXCR4 represents an important feature in the pathogenesis of immunodeficiency and the development of full blown AIDS.

There are five variable regions and five conserved regions that compose gp120 (Starcich, et al., 1986; Wyatt, et al., 1995). Two variable loop regions, V1/V2 and V3, prior to initial viral interaction with the cell surface, are closely associated and block accessibility to a region associated with chemokine receptor binding. Binding of CD4, which occurs above these two variable regions, is dependent upon discontinuous elements in conserved regions 3 and 4 (C3 and C4)(Moore, et al., 1994). Amino acid changes in the V2 and V3 loop regions can alter both the membrane fusion process and HIV-1 tropism (Wyatt, et al., 1995).

Infection of susceptible cells occurs via three conformational stages involving HIV-1 gp120 (D'Sousa et al., 2000). In short, the interaction between HIV-1 and the host cell proceeds as follows: A segment of gp120 binds to CD4 on the host cell surface resulting in an initial conformational change of the V1/V2 and V3 regions of gp120. This change allows access to a portion of gp120, previously covered by the two variable regions, which binds with a co-receptor resident on the host cell. This gp120 conformational change involves movement of the V1/V2 loops away from the V3 loop (Thali, et al., 1993; Wyatt, et al., 1995, Sullivan, et al., 1998). Under normal circumstances, HIV-1 gp120 requires the presence of both the CD4 and a co-receptor to cause additional conformational changes resulting in exposure of gp41. The viral protein, gp41, is responsible for fusion and entry. The CD4 co-receptor is either CXCR4 or CCR5 and is determined by the tropism of the virus (Feng, et al., 1996; Doranz, et al., 1996; Deng, et al., 1996; Choe, et al., 1996; Wu, et al., 1996). The extracellular portion of gp41 contains two helical domains: HR1 and HR2 (or NHR and CHR; Jiang, et al., 2002). The tip of gp41 inserts into the host cell membrane and anchors the virus to the cell. The two helical domains of gp41, previously separated by a segment of gp120, bind together to form a 6-helix bundle that is a fusogenic structure (Jiang, 2002). The virus and cell surface are pulled together by this structure, allowing fusion of the virus envelope and host cellular membrane and insertion of viral genetic material. The co-receptor CCR5, whose natural ligands are the a chemokines RANTES, MIP-1-a, MIP-1-t and MDC, is employed by primary isolates of HIV-1 which are generally M (macrophage) tropic, and is found on T cells and macrophages. CXCR4, whose natural ligand is SDF-1a, is employed by late stage HIV-1 isolates and is employed by T (T cell)-tropic HIV-1. There is an in vivo switch in tropism during HIV infection (Wyatt and Sodroski, 1998).

Due to the complexity of the binding and penetration of HIV-1, the virus is, at least theoretically, vulnerable to either single or, more especially, multiple entry inhibitors. Therefore, there are several cellular sites and viral sites with which inhibitors could interact to halt the process: CD4, CXCR4, CCR5, gp120 and gp41. The substances currently under consideration generally have high cost in addition to limited production as well as low bio-availability and poor pharmacologic and toxicology profiles. Nineteen potential binding/entry inhibitors were listed in 2000 (D'Sousa, et al., 2000); work is still progressing and a glance at the current literature indicates new additions in the list. Gp41 inhibitors T-20 and T-1249 (Trimeris/Hoffman LaRoche) as well as PRO-542 (Progenics), PRO-2000 (Procept) and Cyanovirin (CV-N) all of which target virus/CD4 interaction and AMD-3100 (AnorMed), which interferes with HIV/CXCR4 interactions, are still viable candidates. These compounds are representative of, and provide an overview of, current thought in the area of inhibiting viral binding/entry (De Clercq, 2002).

The drug candidates listed above suggest that combinatorial efforts to prevent binding and entry is likely to become the norm, as opposed to the use of single drugs, as indicated by the synergistic combination of drugs with T-20. Additionally, the concept of disease prevention by the use of binding/entry inhibitors is established in the research and clinical communities. The use of PRO-2000 in a vaginal gel, coupled with the early results achieved, suggest that this is a potentially viable approach, especially given that this is associated with the most frequent mode of transmission (Greenhead, 2000). This topical approach is strengthened by the determination that HIV must transit the epithelial lining of the vagina wall to access infection susceptible cells, that epithelial cells are not subject to infection and they do not aid transport of the virus. In fact, the epithelial cells may act as a barrier to infection. The presence of PRO2000 was found to result in 97% reduction in HIV infection in an in-vitro cervical explant test system (Greenhead, 2000).

Molecular Mimicry; Alpha-neurotoxin/HIV gp120 Sequence Homology

Death by cobra envenomation is attributed to the interaction of basic polypeptides (cobra alpha-neurotoxins) that act post-synaptically and result in blockade of nerve transmission due to their affinity for the nicotinic acetylcholine receptor (nAchR). nAchRs are ligand-gated ion channels activated by the binding of acetylcholine (Ach). On muscle, the nAchR molecule is a pentamer composed of two alpha subunits, one beta, one gamma and one delta subunit. Ach binds to the alpha subunit, each nAchR complex having two acetylcholine binding sites (Dowding et al., 1987). Cobratoxin and other snake alpha-neurotoxins are curaremimetic since they mimic the actions of curare in that they are potent competitive inhibitors of Ach binding to the nAchR and blocking Ach activity. The action of cobratoxin differs from that of curare and strychnine in that the effects of these two substances in vitro is reversed by washing, while the action of cobratoxin is irreversible. A large number of curaremimetic toxins have been isolated from the venoms of elapid and hydrophid snakes and similar curaremimetic toxins have been isolated from the venom of sea snails of the Conus genera. Overall, the snake proteins have a structural homology, being small proteins with a clover leaf-like shape consisting of three adjacent loops that emerge from a small globular core (LeGoas et al., 1992). The neurotoxin of the cobra of interest, Naja naja kaouthia, is a long chain neurotoxin that is cross-linked with 5 disulfide bonds (LeGoas et al., 1992). Loop one is partly hydrophobic and partly exposed to water, this portion having the greater flexibility. The central, or toxic loop, loop 2, is the largest loop and is mainly composed of two strands from the beta-pleated sheet. This loop bears an amino acid sequence homologous with HIV-1 gp120 and rabies virus glycoprotein (RVG). Loop 3 is closed by a disulfide bond and is nearly perpendicular to the beta sheet plane (LeGoas, et al., 1992). All known potent alpha-neurotoxins contain a single invariant tryptophan residue in the same or similar position in the primary sequence (Chang, et al., 1990). This tryptophan residue occupies amino acid position 28 in alpha-bungarotoxin (Bungarus multicinctus) and position 25 in alpha-cobratoxin (Naja naja kaouthia).

The a-neurotoxins of Naja naja kaouthia (cobratoxin) and Bungarus multicinctus (bungarotoxin) have a sequence homology with HIV gp120 and rabies virus glycoprotein (RVG) as indicated below in Table I. This homology is located in a manner that it is accessible for the production and interaction with antibodies on both viruses. Like the homologous sequence on elapid toxins, the amino acid sequence present in rabies virus glycoprotein (RVG) and gp120 of HIV results in interaction with the nAchR. This interaction has been demonstrated by the binding of rabies virus (Lentz, et al., 1982, Lentz, et al.,1987) and HIV-1 gp120 (Bracci, et al., 1992). Both viral interactions were blocked by the use of -bungarotoxin.

The apparent domain of sequence homology on HIV gp120 is located at amino acid residues 159-169, which places it at the initiation of the loop of the gp120 variable region 2 (V2), and is associated with the V1/V2 loop region.

TABLE I

SEQUENCE HOMOLOGY OF HIV-1, RVG and SNAKE
NEUROTOXINS

| | |
|---|---|
| RVG (189-199) | C D I F T N S R G K I |
| HIV-gp120 (159-169) | F N I S T S I R G K V |
| Peptide B2 | S F N I S T S I R G K V Q I |
| -cobratoxin (30-40) | C D A F C S I R G K R |
| -bungarotoxin (30-40) | C D A F C S S R G K [symbol] |

From Neri et. al.; 1990; Bracci et. al.; 1992; Bracci et. al.; 1997, Meyers and Lu; 2002

The apparent domain of sequence homology on HIV gp120 is located at amino acid residues 159-169, which places it at the initiation of the loop of the gp120 variable region 2 (V2), and is associated with the V1/V2 loop region.

There are five variable regions and five conserved regions on gp120 (Starcich, et al., 1986; Wyatt, et al., 1995). Binding of CD4 is dependent upon discontinuous elements in conserved regions 3 and 4 (C3 and C4) while the V3 and V4 regions are the most exposed elements of the multimeric envelope glycoprotein complex (Moore, et al., 1994). Changes in the V2 and V3 loop regions can alter both the membrane fusion process and HIV-1 tropism (Wyatt, et al., 1995).

The sequence homology existing between gp120 and snake a-neurotoxins is not obviously associated with the host cell CD4 binding, in the context of a known receptor sequence on the CD4 molecule. Thus there does not appear to be an obvious association between the sequence and viral interaction with potential host cells, given the currently accepted binding/entry scenario. With respect to that scenario, as indicated previously, there are considerable viral conformational alterations associated with the CD4-gp120 interaction. Binding thermodynamics, as reported by Myszka, et al; (2000), are of unexpected magnitude and indicative of extensive structural rearrangements. One of these rearrangements is the movement of the V1/V2 loops which results in the exposure of the conserved discontinuous structures which are recognized by monoclonal antibodies (Thali, et al., 1993; Wyatt, et al., 1995; Sullivan, et al., 1998). Conformational alterations of the V1/V2 loop structures also result in exposure of the site for interaction with the CCR5 chemokine receptor (Kolchinsky, et al., 2001). It has been suggested, based upon an induced mutation of the a3 strand of the bridging sheet between V1/V2 and V3 (Zhu, et al., 2001), that there is a direct interaction between V1/V2 and V3. Since the V2 loop gp120 site is exposed on an aspect of the protein that interacts with the potential host cell (Wyatt and Sodroski, 1998), and the demonstrable presence of nAchR on CD4+ cells, there is a possibility that a natural reaction with HIV-1 with nAchR occurs. The ability of HIV-gp120 to bind to the nAchR as well as the proven capability of modified neurotoxin to bind to the same receptor permits the hypothesis that modified neurotoxins may act as an entry inhibitor particularly in the nervous system.

The Presence of nAchR on CD4+ Cells.

A better and more documented rational for modified neurotoxins, potential as HIV-1 entry inhibitors of lymphocytes is in the interaction of the homologous a-neurotoxins with nAchR present on CD4+ cell surfaces. Human “T” lymphocytes are a major source for acetylcholine (Ach) (Fujii and Kawashima, 2001; Sato, et al., 1999; Kawashima, et al., 1998; Fujii, et al., 1996). Additionally, there is a substantial body of work indicating the presence of both muscarinic AchRs (mAchRs) and nicotinic AchRs on the surface of human peripheral blood mononuclear cells (PBMC) (Fujii and Kawashima, 2001; Singh, et al., 2000; Kawashima and Fujii, 2000). Messenger RNA expression of subunits for both nAchR (a2-a7 and a2-a4) and mAchR (m1-m5) was determined for human PBMC indicating the presence of AchR on the cell surface (Sato, et al., 1999). Others (Battaglioli, et al, 1998) have determined the presence of the nAchR a3 promoter in T lymphocytes. Stimulation of T lymphocytes with the mitogen phytohemagglutinin (PHA) results in increased synthesis and release of Ach as well as an increase in mRNA encoding for nAchR and mAchR (Kawashima and Fujii, 2000; Fujii and Kawashima, 2001) and suggests an autocrine and/or paracrine function for Ach in the regulation of immune function (Fujii and Kawashima, 2001). Inhibition of Concanavalin-A (Con A) induced T cell proliferation is blocked by the nAchR antagonist mecamylamine (MEC) and by acute nicotine exposure (Singh et al., 2000). Acute nicotine exposure of ConA stimulated mouse splenocytes resulted in decreased production of IL-10 and also resulted in increased production of IFN-gamma (Hallquist, et al., 2000). The presence of human lymphocyte cell surface nAchRs has been determined by the binding of fluoresceine isothiocyanate (FITC)-conjugated a-BTX; affinity purification of a-BTX bound protein indicated that the nAchR bound were the same as those found in muscle (Toyabe et al., 1997). Additionally, a monoclonal antibody (MoAb), designated as W6, competes with Ach for binding with a-BTX for the Torpedo nAchR a1 subunit (McLane et al., 1992). MoAb W6 mediated immuno-staining indicated the presence of nAchR on the surface of human PBMC which was situated in the perinuclear/surface region and which resembled the binding of antibody specific for CD4+ (Hiemke et al., 1996). The presence of surface a3 and a4 nAchR subunits was determined on human PBMC (Hiemke, et al., 1996) and studies by Benhammou, et al. (2000) using nicotine binding and determination of mRNA expression in PBMC also indicated the presence of a4-a3 and a3-a4 nAchRs. Others have determined the binding of ³H-nicotine to human PBMC indicating the presence of nAchR on the surface with a calculated density of ˜2000 sites/cell (Grabczewska, et al., 1990). Additionally the binding of ³H-nicotine to human neutrophils, monocytes and lymphocytes (Davies, et al., 1982) has been observed. The formation of E-rosettes, a function of T cells from peripheral blood, and a method used for T cell enumeration, is decreased by 30%-40% in the presence of carbamylcholine chloride, a cholinergic antagonist, indicating the expression of nAchR on at least a subset of human T cells (Mizuno, et al., 1982).

Therefore the target receptor for venom alpha-neurotoxins are readily expressed in a variety of cells that can also be infected with HIV. However, studies with other viruses have shown that native alpha-cobratoxin does not have any antiviral activity against either herpes or Semliki Forest virus. Formalin or heat denatured venom or cobratoxin, respectively, also displayed no antiviral activity while the heat-denatured CTX (resulting in beta elimination at the disulphide bonds as measured by mass spectromtry) was still capable of binding to its native receptor. Also, inhibition of viral infection, as by the rabies virus, could be observed in cells devoid of NAChR (BHK-21). Therefore it seemed unlikely that the NAChR receptor was part of the antiviral mechanism. Thus, the type of chemical modification is important to the activity of the final product.

### Production Techniques

Administration of a highly toxic substance such as cobratoxin for therapeutic purposes is fraught with obvious difficulties, even when highly diluted. As a diluted substance, its potential effectiveness is reduced, and due to its high affinity for the nAchR, continued use could result in accumulation of the toxin at neuromuscular junctions and the diaphragm with the potential for adverse events. Alpha cobratoxin, of the Thailand cobra, Naja naja kaouthia, is a homogeneous non-glycosylated polypeptide composed of 71 amino acids with a molecular weight of 7821d and a pI of 9.6. Detoxification of alpha-cobratoxin can be achieved by exposure to heat, formamide, hydrogen peroxide, perchloric acid, ozone or other oxidizing agents. The result of exposure of cobratoxin to oxidizing agents is modification of amino acid side chains as well as the lysis of one or more disulfide bonds. Tu (1973) has indicated that the curaremimetic alpha-neurotoxins of cobra and krait venoms loose their toxicity upon either oxidation or upon reduction and alkylation of the disulfide bonds. The procedures used for detoxification described here are based upon the work of Sanders, who preferred the use of hydrogen peroxide (Sanders, et al., 1975). Loss of toxicity by oxidized alpha-neurotoxins (MCTX), as cobratoxin, can be determined by the intraperitoneal (IP) injection of excess levels of the modified protein into mice. In general, injection of 1.5 mcg of natural cobratoxin will result in the death of a 25 g mouse within 25 minutes. After detoxification, IP injection of a 200 mcL volume of 10 mg MCT/mL is non-toxic. This represents at least a 1300 fold reduction of toxicity.

Alternatively, an enzyme linked immunosorbant assay (ELISA) can evaluate loss of toxicity, as well as potential potency in terms of continued ability to bind to the nAchR. Although detoxified cobratoxin has lost a considerable proportion of its affinity for the nAchR, sufficient affinity remains such that it can be detected by an ELISA. This enables a measurement of the depression in binding of the modified neurotoxin to nAchR, indicative of loss of toxicity, while simultaneously indicating a continued ability of the modified toxin to bind to the nAchR providing a measure of potency (Raymond; unpublished data). To test the effectiveness, or potency of detoxified venom, Sanders utilized a plaque assay with Semliki Forest virus (Miller, et al., 1977).

Sanders applied detoxified cobra venoms to the treatment of polio (Sanders, et al., 1953, 1954a, 1954b, 1958a, 1958b) in primates and amyotrophic lateral sclerosis (ALS) (Sanders and Fellows 1974, 1975, 1978) over a 14-year period under an FDA approved IND. Sanders based his work around the observations of Lamb and Hunter (1904) who demonstrated central nerve cell destruction following Naja naja venom exposure. Sanders postulated the notion of steric interference and/or molecular mimicry where detoxified neurotoxins would have the similar access to the CNS and be capable of blocking nerve cell receptors rendering them unavailable for involvement by deleterious neuro-invasive bacteria, viruses or proteins. Thus, the progression of degenerative neurological diseases could be halted or their progression slowed allowing the immune system time to resolve the disease state.

In a preferred embodiment, the method of the present invention is used to prepare inactivated forms of venoms or neurotoxins, and more preferably neurotoxins listed in the group below.

| | |
|---|---|
| Snake Venoms | Naja sp., Bungarus sp., Ophiophagus sp., |
| | Hemachatus sp., Boulengeria sp., Pseudohaje |
| | sp., Walterinnesia sp., Dendroaspis sp., Elaps |
| | sp., Acanthophis sp., Notechis sp., Oxyuranus |
| | sp., Pseudechis sp., Pseudonaja sp., Aipysurus |
| | sp., Astrotia sp., Enhydrina sp., Hydrophis |
| | sp., Lapemis sp., Laticauda sp., Pelamis sp., |
| Other venom | Conus sp. |
| a-Neurotoxins | -cobratoxin, -cobrotoxin, -bungarotoxin, |
| | erabutoxin, -conotoxins and muscarinic |
| | anticholinergic proteins, M1, M2 and M3. |

Recombinant techniques may prove useful in the production of this antiviral peptides. The cloning of a variety of neurotoxins have proven successful though the majority of efforts have focused upon those toxins which are found only in low quantities in native venoms (Fiordalisi, et al., (1996) Toxicon 34, 2, 213-224, Krajewski, et al. (1999) “Recombinant m1-toxin” presented at the 29^{th} Annual Meeting of the Society for Neuroscience) and also with the desire to produce mutants to study structure/function relationships (Smith, et al., (1997) Biochemistry, 36, no. 25, 7690-7996. Cobratoxin has been cloned (Antil S., Servent D. and Menez A. J. Biol. Chem. (1999) Dec 3; 274(49): 34851-8)[.] Although cobratoxin is abundant and easily obtained from natural sources, in order to study the effect of mutations on its interactions with the acetylcholine receptor, specific recombinant production is desirable. Several bioengineered variants have been proposed by the author who was a contributor to the Smith, et al. (1997) paper which replace the residues required for disulphide bond formation with other residues so as to closely mimic the effects of chemical modifications. As these amino acid substitutions must be expressed in-vivo the availability of modifications are limited to the use of native residues (the standard 20 naturally occurring amino acids) and the host to be employed for expression. In the host the codon usage will be important in ensuring efficient and maximal expression of the novel protein. Theoretically any amino acid can be substituted for cysteine but as this is a more costly approach to generating cobratoxin variants relative to synthetic peptide techniques certain residues have been selected which best reproduce the protein characteristics resulting from chemical exposure. It is usual in this circumstance to make what are considered to be conservative substitutions. As a result, it has been chosen to initially limit the cysteine replacement to the following residues; methionine (M), glutamic acid (E), aspartic acid (D), glutamine (Q), asparagine (N), serine (S), glycine (G) and alanine (A). Methionine incorporation would could be considered to be the more conservative substitution by, replacing one sulphur-containing residue for another. Unlike cysteine, methionine cannot form disulphide bonds. Methionine also reacts readily with oxidizing agents to produce the sulfone derivative therefore the purified product can be exposed to chemical agents to confer upon the protein other desirable properties (i.e., low immunogenicity). Also the presence of methionine also allows for the cleavage of the protein into fragments employing cyanogen bromide. Cleavage of the native cobratoxin and modified protein is easily achieved with serine proteases (i.e., trypsin) but at sites containing positive residues. This permits also the evaluation and production of smaller peptide fragments for biological activity (Hinmann, et al., 1999). The conversion of cysteine to cysteic acid also argues for the substitution by other acidic residues such as E, D, Q, N and S. The substitution of E and D for cysteine is estimated to produce a protein with a pI similar to that of modified cobratoxin (pI=4.5). The substitution of cysteine with the residues glycine and alanine would represent standard “neutral” substitutions. The method for creating these genes has been described previously (Smith, et al., 1997). The codon usage of the DNA fragments is optimized for use in commercially used bacterial and yeast expression systems Escherichia coli and Pichia pastoris respectively.

Current technology has also allowed for the production neurotoxins through peptide synthesis. Many smaller neurotoxins (from conus snails, bee venom and scorpion venom) are routinely produced by synthetic peptide methodology (Hopkins, et al., (1995) J. Biol. Chem., 270, no. 38, 22361-22367, Ashcom and Stiles, (1997) Biochem. J. 328, 245-250, Granier, et al., (1978) Eur. J. Biochem., 82, 293-299 and Sabatier, et al., (1994) Int. J. Pept. Protein Res., 43, 486-495) and some are available from commercial organizations. The above references also describe the synthesis of such peptides incorporating mutant residues (Hopkins, et al. (1995) and Sabatier, et al (1994)). Current techniques in peptide chemistry allow for proteins in excess of 80 amino acids can be reliably produced using automated Fmoc solid phase synthesis (ABI 433A Peptide Synthesizer, Perkin Elmer—see www.perkin-elmer.com). Non-native amino acids (acetamidomethyl cysteine, carboxyamidomethyl cysteine, cysteic acid, kynurenine and methionine sulphone) can be acquired from Advanced Chemtech (Louisville, Ky.) or Quchem (Belfast, Ireland). Other oxidized or alkylated amino acid variants are available from these agents. The generation of a synthetic version of the neurotoxin can be achieved by substituting primarily the cysteine residues (from 1 pair to all 5 disulphide couples) with those residues described above to mimic the effects of the various chemical modifications. Furthermore the substitution of other native and non-native residues for cysteine can be investigated in an attempt to identify neurotoxin variants with improved biological activity. Also peptide fragments from within the cobratoxin sequence can be created (analogous to Hinmann et al., (1999), Immunoparmacol. Immunotoxicol., 21 (3), 483-506) and examined for receptor binding activity.

To inhibit infection of cells by HIV in vitro, cells are treated with the MCTX of the invention, or a derivative thereof, either prior to or concurrently with the addition of virus. Inhibition of infection of the cells by the MCTX of the invention is assessed by measuring the replication of virus in the cells, by identifying the presence of viral nucleic acids and/or proteins in the cells, for example, by performing PCR, Southern, Northern or Western blotting analyses, reverse transcriptase (RT) assays, or by immunofluorescence or other viral protein detection procedures. The amount of MCTX and virus to be added to the cells will be apparent to one skilled in the art from the teaching provided herein.

To inhibit infection of cells by HIV in vivo, the MCTX of the invention, or a derivative thereof, is administered to a human subject who is either at risk of acquiring HIV infection, or who is already infected with HIV. Prior to administration, the MCTX, or a derivative thereof, is suspended in a pharmaceutically acceptable formulation such as a saline solution or other physiologically acceptable solution which is suitable for the chosen route of administration and which will be readily apparent to those skilled in the art of MCTX preparation and administration.

Typically, the MCTX is administered in a range of 0.1 mcg to 2 mg of protein per dose. Approximately 1-10 doses are administered to the individual at intervals ranging from once per day to once every few years. The MCTX may be administered by any number of routes including, but not limited to, subcutaneous, intramuscular, oral, intravenous, intradermal, intranasal or intravaginal routes of administration. The MCTX of the invention may be administered to the patient in a sustained release formulation using a biodegradable biocompatible polymer, or by on-site delivery using micelles, gels and liposomes, or rectally (e.g., by suppository or enema). The appropriate pharmaceutically acceptable carrier will be evident to those skilled in the art and will depend in large part upon the route of administration.

### EXAMPLES

### Example 1

Venom Modification

Venom from the Thailand cobra (Naja naja kaouthia) was purchased from Biotoxins (Florida) or Kentucky Reptile Zoo (Kentucky). Employing the procedure described by Sanders (U.S. Pat. No. 3,888,977) and Miller, et al. (1977) the reactive molecule, hydrogen peroxide, the precursor protein is modified through the addition of oxygen molecules.

Other venoms detoxified in this manner include venoms from Naja naja atra, Bungarus multicintus, and Crotalus durissus terrificus.

### Example 2

Neurotoxin Modification

Cobratoxin (CTX) has a molecular weight of 7821 and is composed of 71 amino acids. Alpha-cobratoxin from the Thailand cobra (Naja naja kaouthia) was purchased from Biotoxins, Kississimi, Florida. Employing the procedure described by Sanders (U.S. Pat. No. 3,888,977) and Miller, et al. (1977) the reactive molecule, hydrogen peroxide, the precursor protein is modified through the addition of oxygen molecules.

A modified neurotoxin (MCTX) solution has an acidic pH and a pI of approximately 4.5. Cobratoxin solutions are basic having pH of [10.4]. 8.5. In solution, the drug migrates through molecular sieving gels as monomers, dimers and tetramers. Cobratoxin migrates under these conditions as a monomer. Upon analysis on NuPAGE (Stratagene) SDS polyacrylamide gel electrophoresis (PAGE) the cobratoxin migrates as a 14 Kd and 8 Kd protein with a reference to comparable proteins under unreduced and reduced conditions respectively. MCTX migrates under reduced and unreduced conditions without change. A single protein band is not obtained showing a diffuse smear from the loading gel down to a molecular weight equivalent to 8 Kd. Additionally, the protein is resistant to staining with standard coomassie dyes. By ion exchange, cobratoxin and MCTX have generally opposite properties consistent with the proteins' charges. Specialized ion-exchange chromatographic resins and conditions can be employed to confirm the retention of positive charges which are considered critical for neuroactive properties.

As defined by mass spectrometry the molecular weight of MCTX both purified and in venom is 6,777 to 8,000 daltons. Smaller than expected molecular weights suggest protein fragmentation or side chain modifications. Smaller than expected molecular weights suggest protein fragmentation. Current analytical techniques allow for limited structural identification of the number and location of oxidized residues being added to the protein and rely heavily on previously published information and current chemical theory. Amino acid analyzers do not recognize unnatural amino acids and have limited capabilities for this application.

### Example 3

Toxicity Assay in Mice

The endpoint of the above reactions are most easily determined by assessing the toxicity of the preparation in mice. Mice are sensitive to the actions of many venoms particularly to that of snakes. The proven LD50 of pure alpha-cobratoxin in mice is 1.2 mcg with death observable within hours when injected subcutaneously or intraperitoneally. If the animal survives overnight it is accepted that the material is not lethal and defines the endpoint of the assay. By administering the composition of the invention at set periods a reduction in the material's toxicity can be observed as an increase in time to death. When 5 mg of the protein solution can be administered without inducing death then the reaction process is complete. This represents more than a 4000 fold reduction in toxicity. It is at this point that the solution takes on its antiviral properties and native cobratoxin does not demonstrate antiviral activity in similar assays.

### Examples 4

Antiviral Experiments with Modified Venom and Neurotoxin.

Based upon findings that modified snake alpha-neurotoxins have lymphocyte chemotaxic functions, as well as an observed amino acid sequence homology between HIV-1 gp120 and cobratoxin, the ability of oxidized venom and the purified alpha-cobratoxin to block in vitro HIV-1 infection in a thymus explant system and in PHA stimulated PBMC was examined. PHA stimulated PBMC were infected with a TCID₅₀ of 200 and 1000 of virus (R5 isolate HIV-1_{Bal} or X4 isolate HIV-1_{Lai}).

Both formulations demonstrate inhibition of the virus. However, the crude venom preparation unexpectedly demonstrated a higher inhibitory activity than that of the purified neurotoxin.

As a generalized procedure for the two laboratories involved in the in vitro testing of oxidized purfied alpha neurotoxin and oxidized venom, the following was performed: PBMC from fresh, HIV-1 non-infected buffy coat cells obtained from healthy donors at local blood banks were purified by the Ficoll method. The buffy coat cells were maintained at room temperature until centrifugation. Purified PBMC were re-suspended at 1E6-3E6 cells/mL RPMI medium supplemented with 10% human AB serum and immediately treated with 5 ug PHA/mL suspension. Two to three days later, cells were counted and used for examination of infection. As a standard procedure, cells were incubated in propagation media, consisting of RPMI media supplemented with 10% human AB serum and 50 units IL2/mL, at a density of 6E6 cells per mL and incubated with 200-1000 TCID₅₀ HIV-1/mL×10E6 PBMC. Infection was allowed for 2 hours at 37° C. and the unbound virus was washed away by two washes with propagation media. 200,000 cells were suspended in 180 uL of propagation media and placed in 96 well plates (U bottom). Twenty uL of a 10× stock of the corresponding dilution of the drug was added to each well. Infections were performed in triplicate and controls containing 1 uM AZT were run in parallel as controls to confirm the validity of the assay. The cultures were incubated at 37° C. for 4 days. At that time, 90 uL of media was removed and replaced with 100 uL of propagation media containing the corresponding dilution of drug. The amount of p24 accumulated in the culture was estimated 3 days later (7 days post infection) with a Becton-Dickenson p24 ELISA. Routinely, a few samples were chosen and 10E-2 to 10E-4 dilutions of culture supernatant were prepared to estimate the linearity of the assay.

### Example 5

Preliminary Studies in Patients with HIV by Parenteral Administration.

Based upon the broad antiviral activity of the modified cobra venoms and the purified alpha-cobratoxin concomitant with the proven safety data in prior human trials a preliminary study was undertaken.

Twenty (20) HIV positive patients volunteered to undergo treatment with the oxidized alpha-cobratoxin in addition to ten (10) HIV negative individuals over a period of 6 months. The modified cobratoxin was their sole therapy regime. Given the severity of the disease in this patient cohort no HIV positive placebo was examined. The drug was administered initially at 1 mcg per day (drug format was 10 mcg/ml in 0.9% saline) increasing daily in 0.1 cc increments to 10 mcg/day and subsequently rising to 20 mcg/day (administered as 1 cc b.i.d.). The participants were supplied with insulin-type syringes and taught to self-administer the drug. The participants presented themselves regularly for blood draws. Full blood analysis was undertaken and the data recorded.

No adverse events were reported in normal patients. General responses in HIV positive patients were good with one reported adverse event in a French female aged approximately 28 who was unavailable for follow-up investigations. Notable observations within 2-3 weeks of treatment were improved energy and strength, improved appetite and cessation of diarrhea episodes. Several patients were noted to have increased in weight by over 15 pounds. General activity increased with several patients returning to full employment.

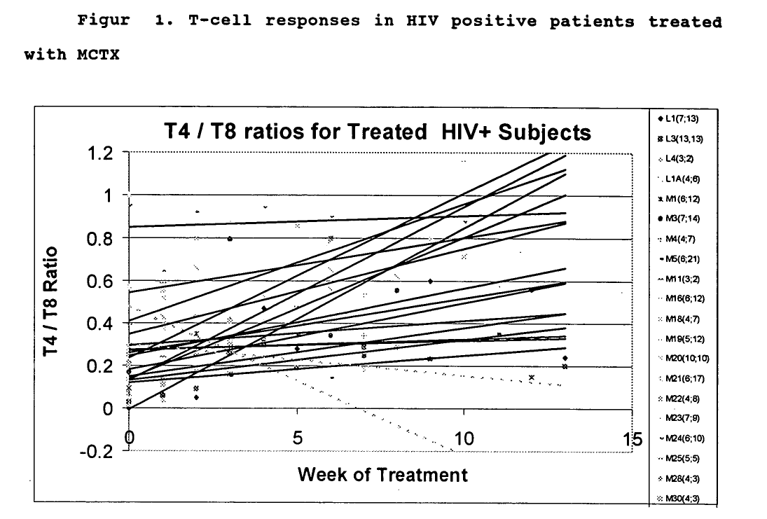

The T4/T8 ratios were recorded and reported in FIG. 1. In normal individuals the ratio is 1. The curves presented represent a least squares linear regression of the available data for each individual over the period of testing for that individual. Overall the general trend of the ratios was to increase over the course of treatment in the majority of HIV-1 positive patients.

### Example 6

Preliminary Studies in Patients with HIV by Oral Administration.

Seven individuals self-administered the MCTX using a buccal spray composed of 600 mcg/mL saline. The protocol provided for the administration of the drug at 0.1 ml seven times per day giving a maximum drug level of 0.7 ml (600 mcg/ml)×50% (efficiency of oral delivery)=210 mcg per day over the course of 3 months. Data obtained for this study suggest MCTX had a noticeable effect in three areas: The percentage of HIV-1 infected T cells, the percentage of HIV-1 infected monocytes and percentage Plasma Viral Load. In all three cases, there was a general trend in the majority of patients toward a decrease in infected cells and plasma viral load, some by as much as 40%.

1. A method of treatment of animals suffering from retroviral infections comprising administering to the animal a disease mitigating amount of a detoxified modified venom composition containing alpha-neurotoxins.

2. The method of claim 1 wherein the detoxified modified composition comprises a fraction of the whole venom containing the alpha-neurotoxins.

3. The method of claim 1 wherein the alpha-neurotoxins are selected from the group consisting of alpha-bungarotoxin, alpha-cobratoxin, alpha-cobrotoxin, alpha-conotoxins (G1, M1, S1, S1A, ImI), alpha-dendrotoxin and erabutoxin.

4. The method of claim 1 wherein the alpha-neurotoxin composition comprises alpha-cobratoxin.

5. The method of claim 1 wherein in humans a dosage of the composition ranges substantially from 0.01 to 10 ml based on a 0.1% solution of the modified cobratoxin per 150 lbs body weight.

6. The method of claim 5 wherein the dosage is from 0.2 to 2 ml.

7. The method of claim 5 wherein the dosage is administered substantially with a frequency of from every other week to daily.

8. The method of claim 5 wherein the dosage is administered at least weekly.

9. The method of claim 5 wherein the dosage is administered at least daily.

10. The method of claim 5 wherein the administration is by one of injection, orally, otically and by intradermal routes.

11. The method of claim 10 wherein administration by injection is by one of subcutaneous, intramuscular and intravenous.

12. The method of claim 1 which further comprises a viral condition benefiting from improved immunological functioning and reduced viral load.

13. The method of claim 1 wherein the retroviral infection is human immunodeficiency virus (HIV).

14. The method of claim 10, wherein the alpha-cobratoxin is administered orally when combined in a solution with benzalkonium chloride.

15. The method of claim 10, wherein the alpha-cobratoxin is administered orally when combined in a solution with benzalkonium chloride at a protein detergent ratio of between 1:6 to 1:8, and preferably 1:7.5.
